(19) United States
(12) Patent Application Publication
(10) Pub. No.: US 2014/0364599 A1
(43) Pub. Date: Dec. 11, 2014

(54) METHOD FOR ACID CATALYZED OLIGOMERISATION OF MONOSACCHARIDES OR DISACCHARIDES

(71) Applicant: STUDIENGESELLSCHAFT KOHLE MBH, Mülheim an der Ruhr (DE)

(72) Inventors: Ferdi Schüth, Mulheim an der Ruhr (DE); Roberto Rinaldi, Mulheim an der Ruhr (DE); Niklas Meine, Mulheim an der Ruhr (DE)

(73) Assignee: STUDIENGESELLSCHAFT KOHLE MBH, Mülheim an der Ruhr (DE)

(21) Appl. No.: 14/366,772

(22) PCT Filed: Dec. 18, 2012
(86) PCT No.: PCT/DE2012/100386

§ 371 Date: Jun. 19, 2014

(30) Foreign Application Priority Data
Dec. 20, 2011 (DE) 10 2011 056 679.1

(51) Int. Cl. C07H 3/06

(52) U.S. Cl. CPC C07H 3/06
USPC 536/123.1

(57) ABSTRACT

A method is disclosed for the acid-catalyzed oligomerization of monosaccharides and/or disaccharides in which monosaccharides or disaccharides are subjected to a mechanical treatment in the presence of an inorganic and/or organic acid. During said process, a catalytic conversion of the monosaccharides or disaccharides takes place.

This application is a 371 of International Patent Application No. PCT/DE2012/100386, filed Dec. 18, 2012, which, in turn, claims priority of German Patent Application No. 10 2011 056 679.1, filed Dec. 20, 2011, the entire contents of which patent applications are incorporated herein by reference.

The present invention relates to a method for the acid-catalyzed oligomerization of monosaccharides or disaccharides, in which monosaccharides or disaccharides or mixtures thereof are brought into contact in the presence of an acid or of an acidic compound or mixtures thereof under the action of mechanical energy with formation of oligosaccharides.

Various methods are known in the prior art in which saccharides are subjected to the action of mechanical energy.

For example, as early as at the start of the 20^{th} century, attempts were made to convert cellulose into smaller molecules by means of mechanical grinding. Ball mills were used in order to reduce the crystallinity of the cellulose. Grohn et al. (Journal of Polymer Science 1958, 551) developed a method for converting cellulose into water-soluble products at a conversion rate of 90%, in which the cellulose was ground in a steel tank for 900 hours.

One procedural approach to catalytically hydrolyzing cellulose is disclosed in WO 2009/061750, in which a method for producing soluble sugars from a cellulose-containing material is described.

A further method is described in the previously unpublished DE 10 2010 052 609, in which cellulose is subjected to a mechanical treatment in the presence of an inorganic and/or organic acid.

Monosaccharides and disaccharides have likewise been subjected to the action of mechanical energy. Steurer et al. (Zeitschrift für Physikalische Chemie, 1944, 193, 248-257) treated glucose and sucrose in a vibratory mill, but even after mechanical treatment for 100 hours, no conversion to other compounds could be established.

On the part of the inventors, it has surprisingly been found that the catalytic conversion of monosaccharides or disaccharides or mixtures thereof in the presence of an inorganic and/or organic acid under the action of mechanical energy leads to the formation of oligosaccharides. On the part of the inventors, oligosaccharides with a number of more than two to up to six units of one monosaccharide were able to be produced in the process. The oligosaccharides here are formed particularly from one type of monosaccharide although different monosaccharides in the chain are also conceivable.

The disaccharide or oligosaccharide preferably comprises aldose units, preferably an aldopentose such as xylose, arabinose, and/or ribose, and/or an aldohexose, such as glucose, galactose and/or mannose.

To carry out the process according to the invention, an inorganic and/or organic acid, and mixtures thereof, can be used. To carry out the method according to the invention, however, the acid can also be selected from acidic, polymeric or inorganic ion exchangers or acidic inorganic metal oxides.

If an organic acid is used when carrying out the method according to the invention, particularly good conversion results can be obtained if the organic acid has a pKa value <3, particularly a pKa value of −5 to 2. Suitable examples are benzenesulfonic acid, p-toluenesulfonic acid, nitrobenzenesulfonic acids, 2,4,6-trimethylbenzenesulfonic acid, and derivatives of benzoic acid, methanesulfonic acid, haloalkanecarboxylic acids such as trifluoroacetic acid, maleic acid, oxalic acid and any desired mixtures of the above organic acids. The acids used should preferably have a pKa value of <2. Preference is given to acids with a pKa value of less than −2.

When carrying out the method according to the invention, good conversion results are also obtained if an inorganic acid with a pKa value <3 is used. Preferably, the pKa value is between −14 and 2. Suitable examples of inorganic acids are mineral acids such as sulfuric acid, hydrochloric acid, phosphoric acid, phosphotungstic acid and nitric acid, with nitric acid being less preferred. It is also possible to use mixtures of the above acids. Preference is given to acids with a pKa value of less than −2.

The inorganic and/or organic acid is used in the method according to the invention in catalytic amounts. Preferably, the inorganic and/or organic acid is used in an amount of from 0.01 to 10 mmol per g of monosaccharide or disaccharide.

To carry out the method according to the invention, the inorganic and/or organic acid can be brought into contact directly, i.e. without use of a solvent according to a type of “dry” catalyzed reaction, with the monosaccharide or disaccharide or mixtures thereof, and then the mixture obtained in this way can be subjected to a mechanical treatment.

However, it is also possible for the inorganic and/or organic acid not to be brought into contact directly with the monosaccharide or disaccharide or mixtures thereof, but instead the monosaccharide or disaccharide is impregnated in a first step with a solution of the inorganic and/or organic acid in a suitable solvent.

This procedure has proven to be particularly advantageous for inorganic acids. For this, the acid is preferably firstly mixed with a suitable solvent. Suitable solvents are all solvents which do not adversely affect the reaction, such as water and organic solvents such as diethyl ether, dichloromethane, ethanol, methanol, THF, acetone and any other polar or nonpolar solvents in which the acid used is soluble, or which permits good mixing of monosaccharide and/or disaccharide and acid in a dispersion, and which has a boiling point of 100° C. and below.

In this step, the solution or dispersion of the inorganic and/or organic acid can be mixed with the monosaccharide and/or disaccharide and, if desired, left to stand for some time. Before the mechanical treatment of the monosaccharide and/or disaccharide, the solvent can be removed again. Particularly if the solvent used is a low-boiling solvent, this can be removed again in a simple manner, either by gentle heating and/or by applying vacuum. The acid, which normally has a higher boiling point, remains on the monosaccharide and/or disaccharide.

The mechanical treatment of the monosaccharide and/or disaccharide can then take place in the presence of the adhering inorganic and/or organic acid. It has been found that the degree of conversion of the monosaccharide and/or disaccharide can be increased by impregnating the monosaccharide and/or disaccharide with inorganic and/or organic acid in the presence of a solvent.

It is also possible to mechanically treat the mixture of monosaccharide and/or disaccharide and acid in a solvent. In this connection, it may be advantageous for the catalytic conversion if the mixture of monosaccharide and/or disaccharide and acid is in the form of a slurry which is then subjected to a mechanical treatment. Such a slurry here can have an only slight liquid supernatant above the volume of the powder mixture, which can in total constitute about 120% by volume of the volume of the powder mixture. In this procedure, the amount of acid used can be reduced.

The mechanical treatment can take place for example by grinding, extrusion or kneading. Mills which can be used are those which comminute the material to be ground using grinding bodies, such as e.g. vibratory mills, stirred mills, stirred ball mills, ball mills etc. Particular preference is given to ball mills. Extruders which can be used are all extruders known from the prior art.

As already reported at the start, conversions of the monosaccharides and/or disaccharides to oligosaccharides of up to 80% can be achieved with the method according to the invention. As a rule, mixtures of water-soluble saccharide dimers and oligomers, such as cellobiose, and monosaccharide are obtained, wherein the formation of byproducts can be largely avoided.

If the method according to the invention is carried out in a ball mill, rotary times of 400 to 1200, preferably 800 to 1000, rpm have proven suitable. The reaction time, i.e. the time in which the mechanical treatment takes place, is usually from 0.01 to 24 hours, with durations from 1.5 to 12 hours being adequate.

In a further embodiment of the method according to the invention, it is possible to modify the resulting oligosaccharides by adding one or more fatty alcohols. For this purpose, preferably one or more fatty alcohols having 8 to 22 carbons can be added to the oligomer mixture as aliphatic, long-chain, monohydric, primary alcohols in an amount up to 10 mol %, based on the monosaccharide or disaccharide. The hydrocarbon radicals here are unbranched and can also be mono- or polyunsaturated. In this way, modified oligosaccharides or mixtures thereof can be obtained.

The oligosaccharides according to the invention or their derivatives modified by fatty alcohols can be used as surface-active agents or concrete additives.

The present invention is explained in more detail in the examples below without limiting the invention to these examples.

### EXAMPLES

### Example 1

A mixture of 1.00 g of D-(+)-glucose (commercial product from Aldrich, USA) and 0.175 g of para-toluenesulfonic acid monohydrate (commercial product from Aldrich, USA) was ground in a steel beaker with steel balls (5 steel balls; individual weight 3.95 g) in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of glucose in the ball mill produced, within a grinding time of 5 hours, a conversion of the glucose to water-soluble products which consist of 63% oligosaccharides, 10% cellobiose and 26% glucose.

### Example 2

A mixture of 1.00 g of D-(+)-cellobiose (commercial product from Fluka, Switzerland) and 0.175 g of para-toluenesulfonic acid monohydrate (commercial product from Aldrich, USA) was ground in a steel beaker with steel balls (5 steel balls; individual weight 3.95 g) in a Pulverisette P7 from Fritsch. The rotary time of the main disk was 800 rpm.

An example of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of cellobiose in the ball mill produced, within a grinding time of 5 hours, a conversion of the cellobiose to water-soluble products which consist of 60% oligosaccharides, 33% cellobiose and 7% glucose.

### Example 3

A mixture of 1.00 g of D-(+)-xylose (commercial product from Fluka, Switzerland) and 0.175 g of para-toluenesulfonic acid monohydrate (commercial product from Aldrich, USA) was ground in a steel beaker with steel balls (5 steel balls; individual weight 3.95 g) in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of xylose in the ball mill produced, within a grinding time of 5 hours, a conversion of the xylose to water-soluble products which consist of 79% oligosaccharides and 21% xylose.

### Example 4

A mixture of 1.00 g of D-(+)-glucose (commercial product from Aldrich, USA) and 0.146 g of benzenesulfonic acid (commercial product from Aldrich, USA) was ground in a steel beaker with steel balls (5 steel balls; individual weight 3.95 g) in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of glucose in the ball mill produced, within a grinding time of 5 hours, a conversion of the glucose to water-soluble products which consist of 71% oligosaccharides, 12% cellobiose and 17% glucose.

### Example 5

A mixture of 0.50 g of D-(+)-glucose (commercial product from Aldrich, USA) and 0.5 g of kaolinite (commercial product from Fluka, Switzerland) was ground in a steel beaker with steel balls (5 steel balls; individual weight 3.95 g) in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of glucose in the ball mill produced, within a grinding time of 10 hours, a conversion of the glucose to water-soluble products which consist of 73% oligosaccharides, 2% disaccharides and 24% glucose.

### Example 6

A mixture of 0.50 g of D-(+)-xylose (commercial product from Fluka, Switzerland) and 0.5 of kaolinite (commercial product from Fluka, Switzerland) was ground in a steel beaker with steel balls (5 steel balls; individual weight 3.95 g, in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) with a mass spectrometer (ESI-MS, Bruker ESQ 3000; high resolution mass determinations: Bruker APEX III FTMS (7 T magnet)).

The acid-catalyzed oligomerization of xylose in the ball mill produced, within a grinding time of 10 hours, a conversion of the xylose to the water-soluble products which consist of 76% oligosaccharides, 3% disaccharides and 21% xylose.

### Example 7

A mixture of 0.50 g of D-(+)-cellobiose (commercial product from Fluka, Switzerland) and 0.5 g of kaolinite (commercial product from Fluka) were ground in a steel beaker with steel balls (5 steel balls; individual weight 3.95 g) in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of cellobiose in the ball mill produced, within a grinding time of 10 hours, a conversion of the cellobiose to water-soluble products which consist of 70% oligosaccharides, 19% disaccharides and 4% glucose.

### Example 8

A mixture of 0.50 g of D-(+)-cellobiose (commercial product from Fluka, Switzerland) and 0.5 g of Amberlyst15 DRY (commercial product from Rohm&Haas, Germany) were ground in a steel beaker with steel balls (5 steel balls; individual weight 3.95 g) in a Pulverisette P7 from Fritsch. The rotary time of the main disk was 800 rpm.

A sample of the resulting solid was dissolved in water and investigated by means of HPLC analysis and GPC analysis. Furthermore, the aqueous solution was investigated after removing the acid by means of solid-phase extraction (SPE cartridge, Chromafix, HR-XA (L), commercial product from Macherey-Nagel) using a mass spectrometer (ESI-MS, Bruker ESQ 3000; high resolution mass determinations: Bruker APEX III FTMS (7 T magnet)).

The acid-catalyzed oligomerization of cellobiose in the ball mill produced, within a grinding time of 5 hours, a conversion of the cellobiose to water-soluble products which consist of 80% oligosaccharides, 12% cellobiose and 8% glucose.

### BRIEF DESCRIPTION OF THE DRAWINGS

The chromatographic and ESI-MS investigations of the reaction products obtained in the examples are shown in FIGS. 1 to 9:

FIG. 1 shows GPC chromatograms (4×TSKgel G-Oligo-PW, 7.8 mm ID×30.0 cm and TSKgel Oligo Guardco; Eluent: Milli-Q® Water, flow rate 0.8 mL min⁻¹) of:
A) with the standards: 1 Maltoheptaose, 2 Maltohexaose, 3 Maltopentaose, 4 Maltotetraose, 5 Maltotriose, 6 Cellobiose, 7 Glucose, 8 Glycerol,
B) Cellobiose 5 h in ball mill,
C) Cellobiose+p-TSA 5 h in ball mill,
D) Glucose 5 h in ball mill, E) Glucose+p-TSA 5 h in ball mill;

FIG. 2 shows an ESI-MS (pos. mode) with a spectrum of the oligomerization products following reaction of glucose with p-TSA in ball mill. The m/z values correspond to the [M+Na]⁺ ions. (Glc: glucose fragment, LG: levoglucosan fragment);

FIG. 3 shows an ESI-MS spectrum (pos. mode) of the oligomerization products following reaction of cellobiose with p-TSA in ball mill. The m/z values correspond to the [M+Na]⁺ ions. (Glc: glucose fragment, LG: levoglucosan fragment);

FIG. 4 shows an ESI-MS (pos. mode) with a spectrum of the oligomerization products following reaction of xylose with p-TSA in ball mill. The m/z values correspond to the [M+Na]⁺ ions. (Xyl: xylose fragment);

FIG. 5 shows an ESI-MS (pos. mode) with a spectrum of the oligomerization products following reaction of glucose with BSA in ball mill. The m/z values correspond to the [M+Na]⁺ ions. (Glc: glucose fragments);

FIG. 6 shows an ESI-MS (pos. mode) with a spectrum of the oligomerization products following reaction of glucose with kaolinite in ball mill. The m/z values correspond to the [M+Na]⁺ ions. (Glc: glucose fragments);

FIG. 7 shows an ESI-MS (pos. mode) with a spectrum of the oligomerization products following reaction of xylose with kaolinite in ball mill. The m/z values correspond to the [M+Na]⁺ ions. (Glc: glucose fragments);

FIG. 8 shows an ESI-MS (pos. mode) with a spectrum of the oligomerization products following reaction of cellobiose with kaolinite in ball mill. The m/z values correspond to the [M+Na]⁺ ions. (Glc: glucose fragments), and FIG. 9 shows an ESI-MS (pos. mode) with a spectrum of the oligomerization products following reaction of cellobiose with Amberlyst15DRY in ball mill. The m/z values correspond to the [M+Na]⁺ ions. (Glc: glucose fragments).

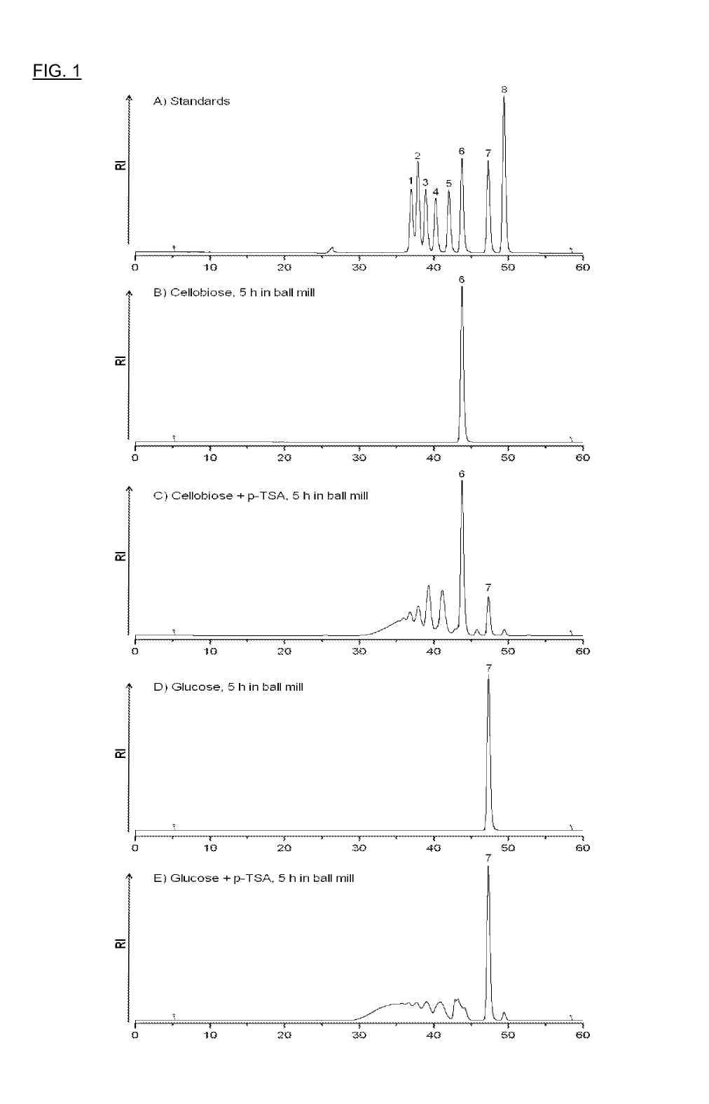

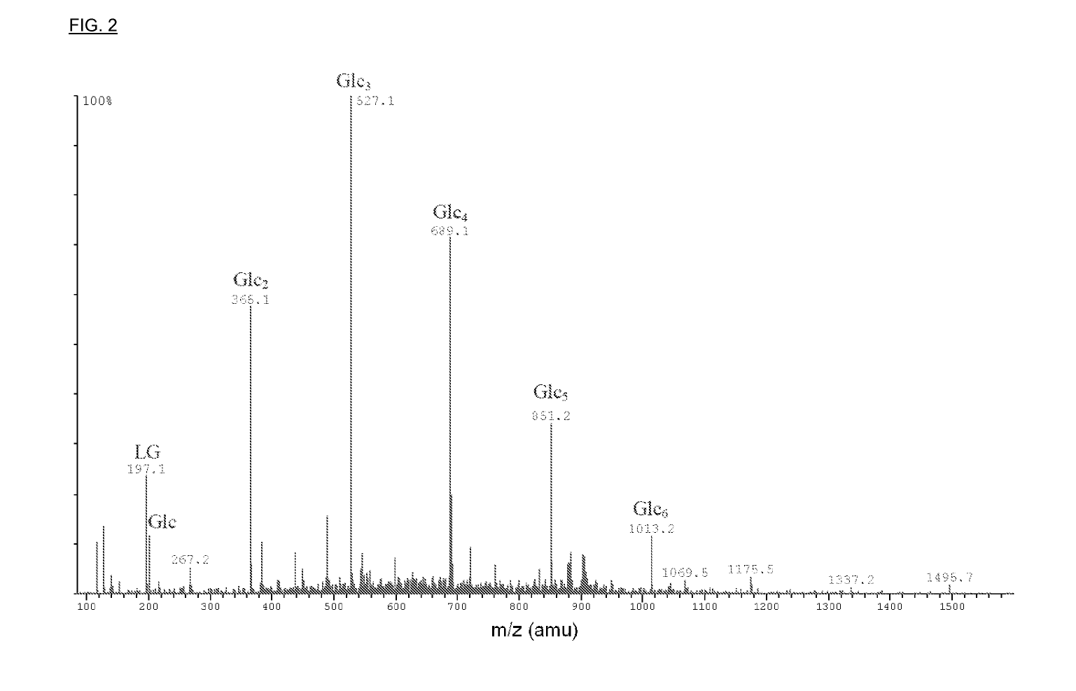

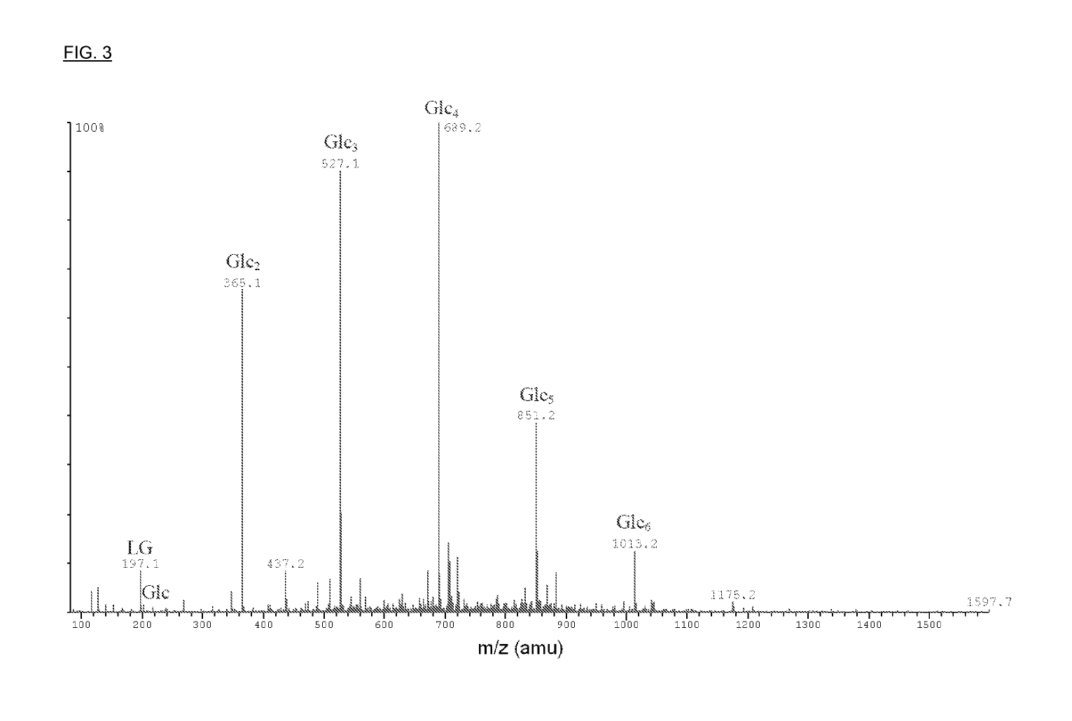

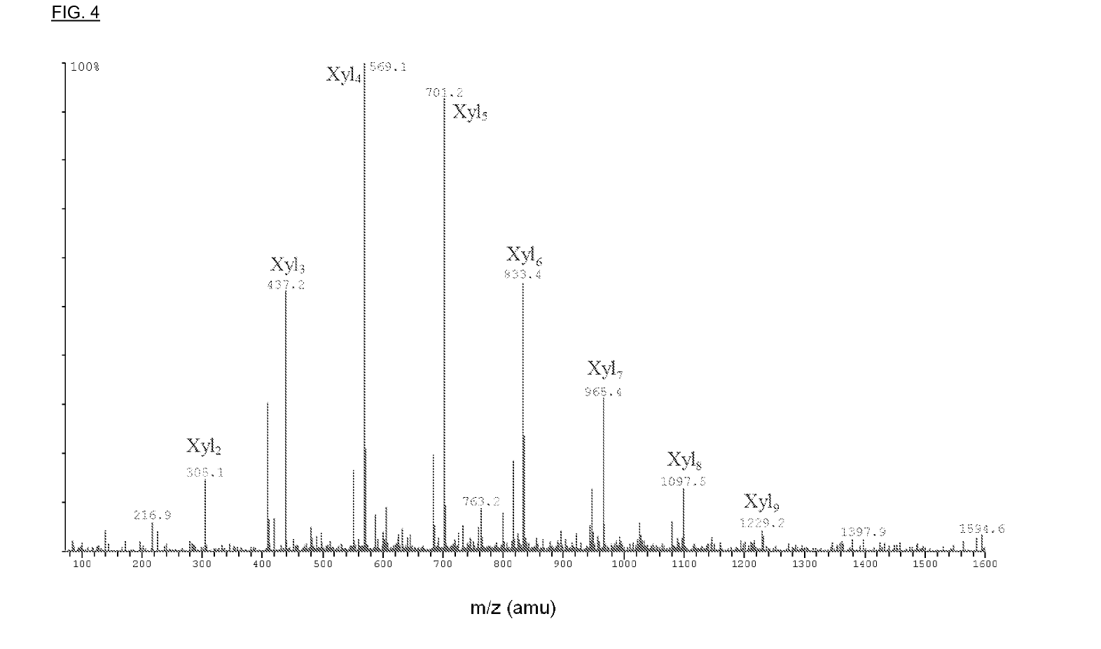

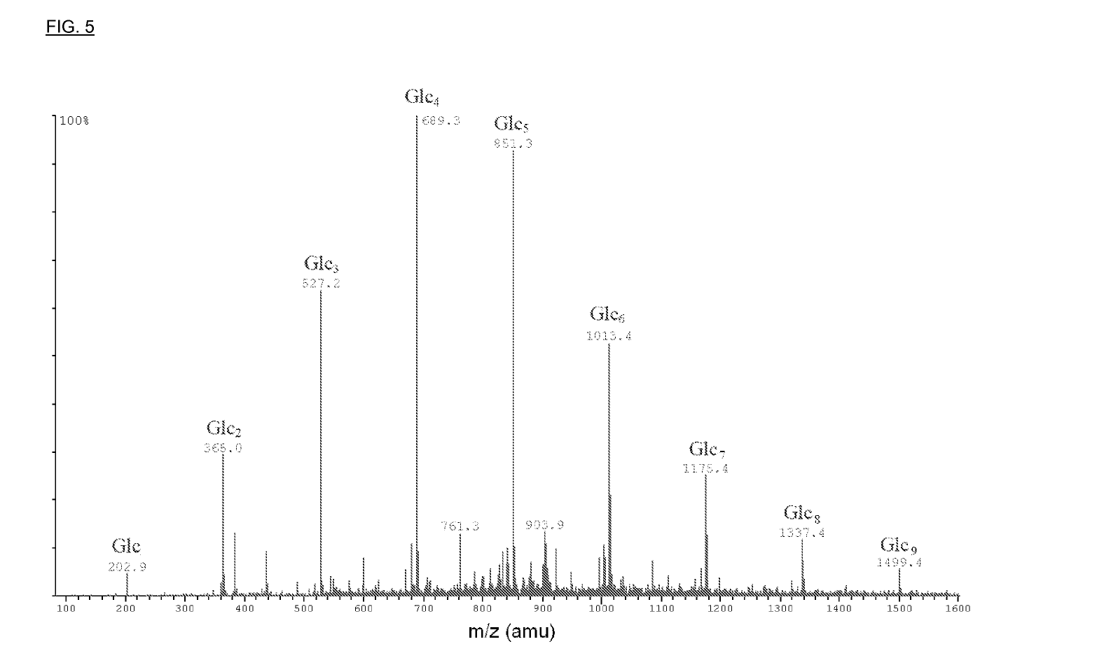

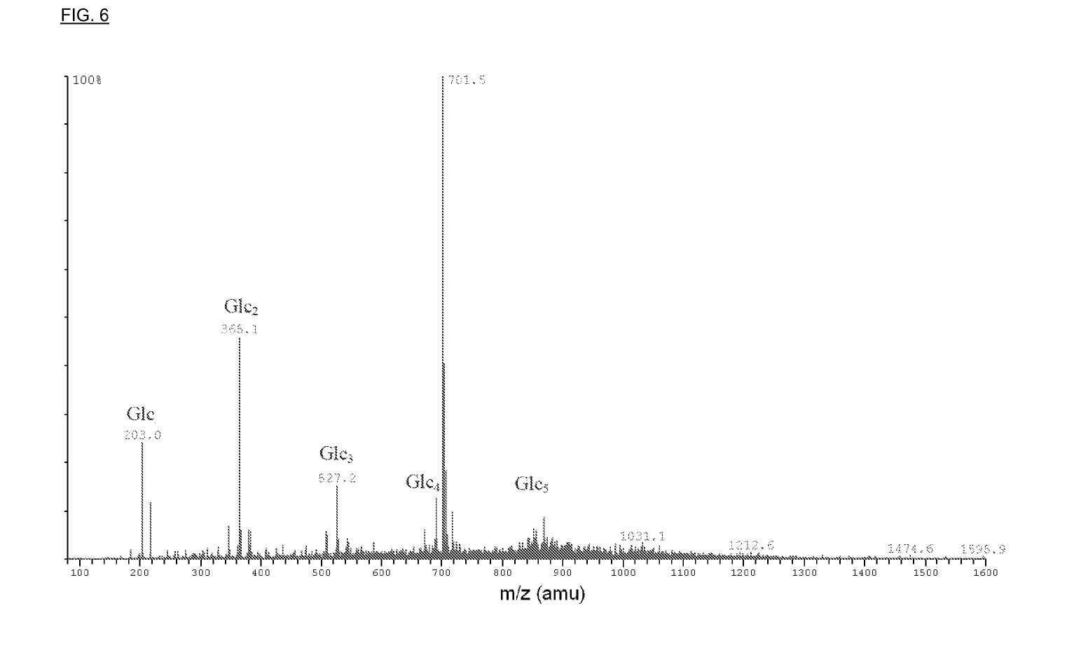

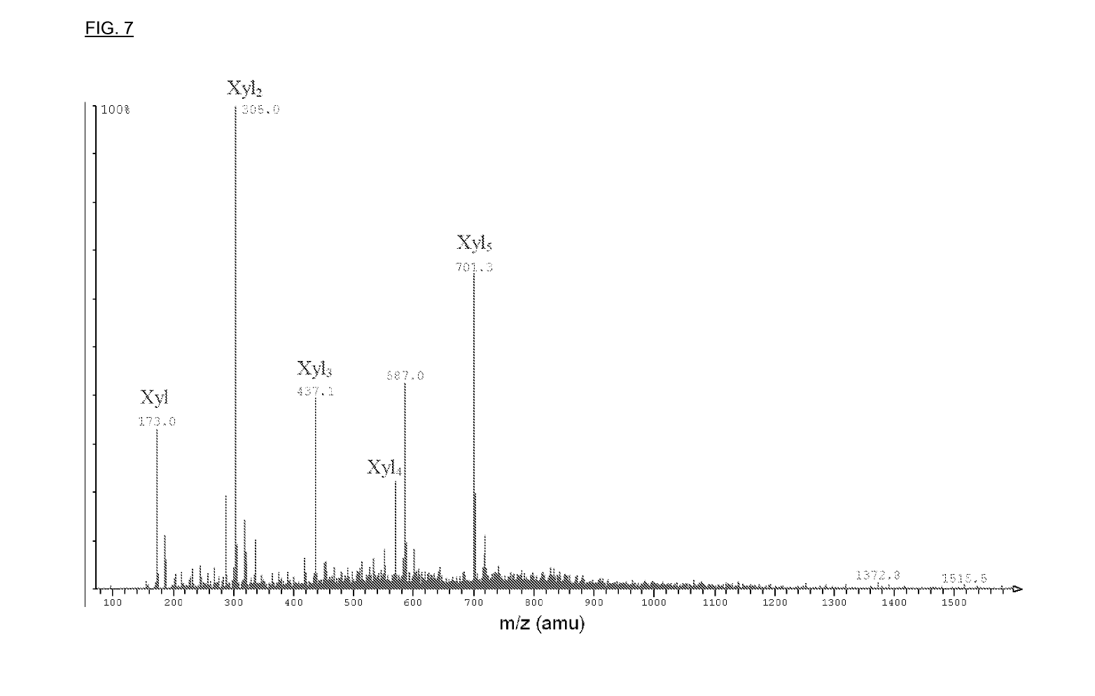

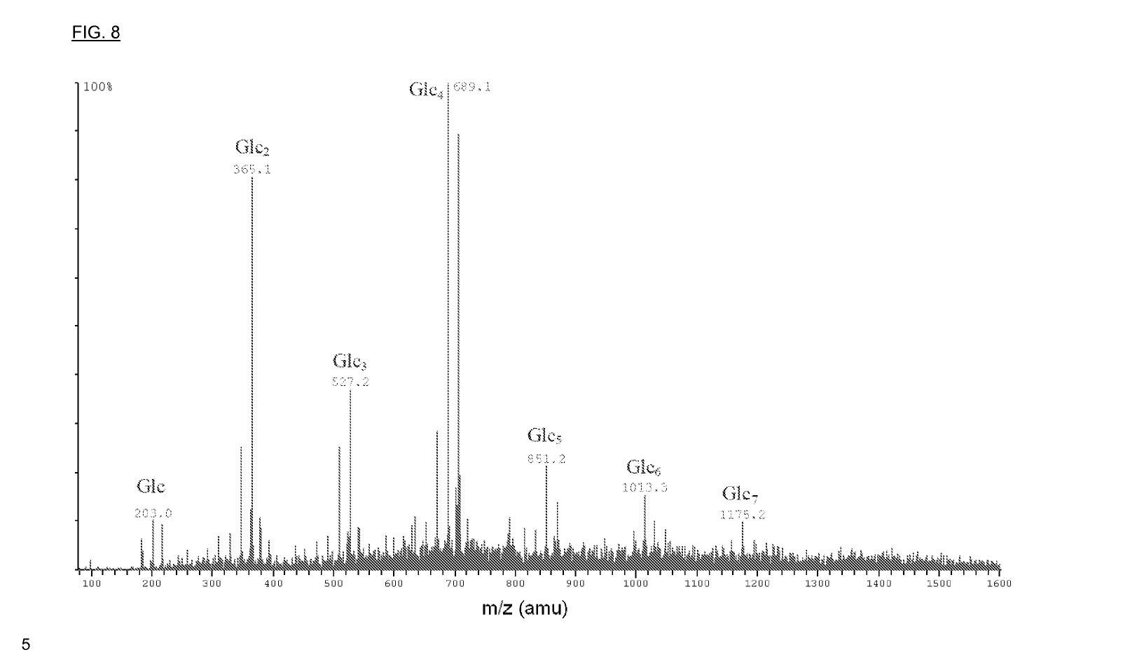

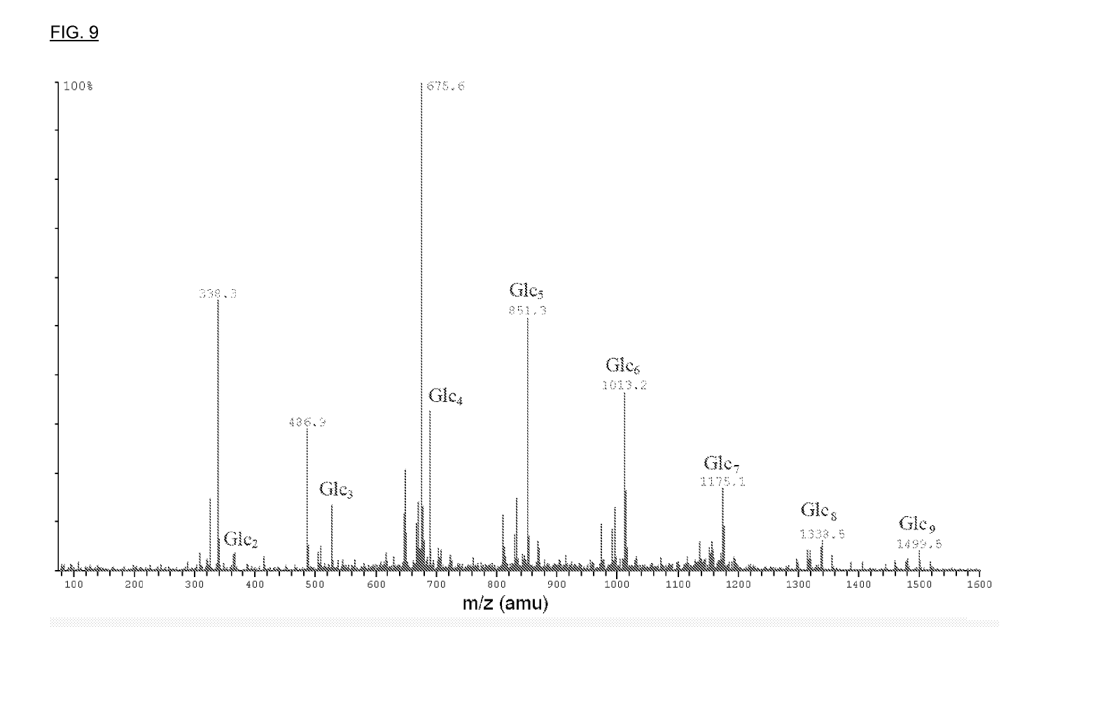

1. A method for the acid-catalyzed oligomerization of monosaccharides and/or disaccharides, said method comprising mechanically treating a monosaccharide and/or disaccharide or mixtures thereof in the presence of an inorganic and/or organic acid.

2. The method as claimed in claim 1, in which the monosaccharide and/or disaccharide is selected from the group consisting of aldopentose or aldohexose, dimers thereof and mixtures thereof.

3. The method as claimed in claim 1, wherein said mechanically treating the monosaccharide and/or disaccharide is in the presence of an inorganic and/or organic acid in the form of a powder mixture or a slurry.

4. The method as claimed in claim 1, wherein the acid has a pKa value of −14 to 2.

5. The method as claimed in claim 1, wherein the inorganic acid is selected from the group consisting of sulfuric acid, hydrochloric acid, phosphoric acid, nitric acid, haloalkanecarboxylic acids phosphotungstic acid and mixtures thereof.

6. The method as claimed in claim 1, wherein the organic acid is selected from the group consisting of benzenesulfonic acids, methanesulfonic acid, maleic acid, oxalic acid and mixtures thereof.

7. The method as claimed in claim 1, wherein the acid is selected from acidic polymeric or inorganic ion exchangers or acidic inorganic metal oxides.

8. The method as claimed in claim 1, wherein the acid is used in an amount of from 0.01 to 10 mmol per g of monosaccharide and/or disaccharide.

9. The method as claimed in claim 1, which further comprises treating the monosaccharide and/or disaccharide before said mechanically treating with the acid or a mixture thereof in a solvent.

10. The method as claimed in claim 9, which further comprises removing the solvent before said mechanically treating.

11. The method as claimed in claim 1, wherein said mechanically treating comprises grinding, in which the material to be ground is comminuted using grinding bodies.

12. The method as claimed in claim 11, wherein said grinding is performed in a mill, and the mill is selected from vibratory mills, stirred mills, stirred ball mills and ball mills.

13. The method as claimed in claim 1, wherein said mechanically treating comprises extrusion or kneading.

14. The method as claimed in claim 1, which further comprises freeing from the adhering acid the mixture comprising the reaction products after the mechanical treatment and, optionally, separating into the individual reaction products.

15. A saccharide oligomer mixture with a degree of oligomerization of 3-8 units for at least 60 mol % of the oligomer mixture.

16. A saccharide oligomer mixture obtainable by the method as claimed in claim 1, said saccharide oligomer having a degree of oligomerization of 3-8 units for at least 60 mol % of the oligomer mixture.

17. A method of using the saccharide oligomer mixture as claimed in claim 15 as surface-active agent.
